(19) United States
(12) Patent Application Publication
(10) Pub. No.: US 2024/0108281 A1
(43) Pub. Date: Apr. 4, 2024

(54) DEVICE FOR DETERMINING TONGUE POSITION BY MEASURING NEGATIVE PRESSURE IN THE ORAL CAVITY, FOR MEASURING INHALATION PRESSURE IN THE NASOPHARYNGEAL CAVITY, AND ASSOCIATED TERMINAL

(71) Applicants: Sandra Vivian KAHN, San Mateo, CA (US); Wilfried Gerhard Hermann ENGELKE, Gleichen (DE)

(72) Inventors: Sandra Vivian KAHN, San Mateo, CA (US); Wilfried Gerhard Hermann ENGELKE, Gleichen (DE)

(21) Appl. No.: 18/274,513

(22) PCT Filed: Jan. 31, 2022
(86) PCT No.: PCT/EP2022/052245

§ 371 Date: Jul. 27, 2023

Related U.S. Application Data
(60) Provisional application No. 63/143,399, filed on Jan. 29, 2021.

(30) Foreign Application Priority Data
Mar. 16, 2021 (ES) P202130232

(51) Int. Cl. A61B 5/00; A61B 5/03

(52) U.S. Cl. CPC A61B 5/4552; A61B 5/038; A61B 5/4205; A61B 5/682; A61B 2560/0214; A61B 2562/0247; A61B 2562/18

(57) ABSTRACT

The invention relates to a device for determining the position of the tongue from the measurement of the vacuum pressure in an oral cavity generated by deglutition, comprising an intraoral shield intended for being arranged in the oral cavity, a conduit connectable at a first end to the intraoral shield and at a second end to a pressure gauge, in turn coupleable to a base, and an electronic module coupleable to the base. The electronic module is in data communication with the pressure gauge and comprises a processing unit, such that in an operating condition of the device, the intraoral shield is configured for blocking interdental spaces in the oral cavity and the conduit establishes fluid communication between the interior of the oral cavity and the pressure gauge, such that the negative pressure generated by deglutition is measured by said pressure gauge.

### TECHNICAL FIELD

The present invention falls within intraoral devices, specifically in the sector of vacuum-activation intraoral devices, and more specifically intraoral devices that facilitate the creation of vacuum pressure in the oral cavity and its measurement based on the tongue movement produced by swallowing.

Likewise, the device of the invention relates to measuring inhalation pressure in the nasopharyngeal cavity.

### BACKGROUND OF THE INVENTION

The need to keep the tongue in the correct position to prevent, for example, loud breathing such as snoring, which occurs when breathing through the mouth during sleep when the tongue partially blocks the airways, is known in the clinical world. For this reason, it is necessary for the tongue to remain in a forward position which ensures that these passages are kept clear or unobstructed.

In addition, apart from the discomfort caused by snoring during the stages of sleep, obstruction of the airways can represent a source of health problems, causing a cut in the air supply to the lungs, commonly known as apnoea. This decreases the amount of oxygen carried by the blood to the brain, causing disruptive sleep patterns and resulting in chronic fatigue that can cumulatively cause damage.

That is why it is necessary for many people to reduce or eliminate these incorrect positions of the tongue inside the oral cavity, avoiding the possible respiratory problems that it may cause.

There are numerous documents in the state of the art that refer to devices intended to correctly position the tongue.

One example of said documents is U.S. Pat. No. 5,373,859, which describes a device for retaining the tongue and reducing snoring, comprising a housing that defines a flexible vacuum reservoir and a rigid flange that completely surrounds the housing and makes contact with the user's face around the entire perimeter of the mouth. The opening of the reservoir fits over the tongue and holds the tongue under negative pressure created with the device.

Document U.S. Pat. No. 4,169,473 also belongs to the state of the art, which discloses a device to be placed inside a user's mouth, with the aim of preventing snoring and/or bruxism, which is configured as a moulded body that provides an externally located portion, and a portion placed inside the user's oral cavity, with dental engaging arches and a rearwardly-opening central socket for cooperating with the forward portion of the tongue, forcing it to be placed in the front portion of the mouth, increasing the unobstructed dimension of the breathing passage.

Document WO2018076088 is also found in the state of the art and proposes an intraoral device for creating a sensation of contact between the teeth, which must be applied to the user's upper and/or lower palate, and which comprises a contact means designed to be associated with at least one portion of one to four posterior teeth, with the aim of creating a sensation of contact for the user by means of the contact between the very contact means and at least one portion of the opposing dental area.

Document ES1067430 also belongs to the state of the art and describes an intraoral device for preventing snoring and/or apnoea, which is made up of a main body, which incorporates a flexible fin on the rear portion thereof, which retains the movement of the tongue, preventing it from moving backwards and therefore preventing the user from snoring.

As can be seen, there are numerous intraoral devices in the state of the art intended to position the tongue inside the oral cavity in such a way that ensures that the user's airways are not obstructed, preventing the tongue from being involved in processes that are harmful to health, such as snoring or apnoea.

However, it has been found that none of the existing systems proposes means for correcting tongue position based on the measurement of the vacuum pressure in the oral cavity generated by swallowing, wherein based on said measured vacuum pressure, a study and/or monitoring of the tongue position can be conducted to assist the user in training to correct an inadequate tongue position and at the same time assist a healthcare professional in understanding the position occupied by the tongue and assessing correction alternatives.

### DESCRIPTION OF THE INVENTION

To respond to the gap found in the technical field, the present invention proposes a device for determining tongue position based on the measurement of vacuum pressure in a user's oral cavity, said negative pressure being generated by the tongue movement during swallowing, to provide information, for example, in the form of a warning to the user or to healthcare personnel about whether a correct tongue position is achieved based on said negative pressure.

It will be obvious to a person skilled in the art that in the context of the invention a negative pressure in the oral cavity refers to a pressure that is lower than the pressure measured outside said oral cavity which generally corresponds to, but is not limited to, the atmospheric pressure at which a user is using the device.

The negative pressure in the oral cavity is generated by swallowing and, to prevent air from leaking into the oral cavity through the dental interstices, by reducing the vacuum pressure; the device of the invention has an intraoral screen, preferably made of a flexible material suitable for non-invasive, intraoral use for the user, which, as mentioned, assists the user in maintaining negative pressure after the swallowing action.

In addition to the intraoral screen, the device comprises a conduit that can be connected by a first end to the intraoral screen at a connection end arranged therein and by a second end to a pressure gauge, in turn able to be coupled to a base of said device. Such that, in a condition of use of the device by a user, the intraoral screen is arranged between the inner portion of the lips and the front portion of the user's teeth to block the dental interstices and the conduit fluidly communicates the inside of the oral cavity with the pressure gauge, such that the negative pressure generated in the oral cavity by swallowing is measured by the pressure gauge.

Preferably, the conduit will have a saliva trap with a membrane made of an impermeable and preferably flexible material, such as GORE-TEX®, which will prevent saliva from entering inside the conduit itself and subsequently contacting the pressure gauge.

Coupled to the base is an electronic module which comprises a processing unit configured to obtain/calculate/determine data on the tongue position based on the measurement of the vacuum pressure in the oral cavity. The electronic module comprises wireless connection means, for example, a Bluetooth card, or a data connection port that can be coupled to the base and connected to said electronic module prepared to transmit data on the pressure in the oral cavity and/or receive data or information to/from any external terminal, such as a computer, smartphone, tablet or similar.

As described above, a device is proposed that assists in the sealing of the oral cavity in order to measure the negative pressure generated by swallowing, for example when swallowing saliva, wherein by measuring the negative pressure it is possible to identify whether the tongue position is correct, and if it not, it can generate signals, transmit data to the external terminal to assist in training the correct position of the tongue, to create habits that prevent the tongue from obstructing the airways.

For example, a negative pressure of 15 m Bar in the oral cavity will indicate that the tongue is in the proper position, occupying the entire oral cavity without any space being able to exist. Therefore, the tongue is fully attached to the palate.

The ideal negative pressure at which the tongue is considered to be in the proper position can be set within a specific range or can be set by medical personnel for a specific user and his or her particular condition.

Thus, with the device it is possible to monitor pressure in the oral cavity and/or changes therein, indicative of the position of the tongue, and said device can also be configured to monitor the time that the negative pressure is maintained in the oral cavity by the user.

In a preferred embodiment, the device comprises means for measuring the vacuum pressure generated by swallowing in an air chamber formed between the dorsum of the tongue and the palate, better known as the space of Donders, wherein such means comprise a flexible tube or cannula that can be connected by a first end to the conduit and/or to the pressure gauge and a second end, opposite the first end, open to the atmosphere, so that in an operating condition of the device, the second end of the tube is arranged in the space of Donders, so that the pressure gauge detects the air pressure in said space of Donders and sends the data on said detection to the processing unit for subsequent processing of such data.

A person skilled in the sector will know that the space of Donders is a virtual space that is generated with respect to the hard palate behind the lip seal and up to the seal between the dorsum of the tongue and the soft palate. A negative pressure is generated in this space, capable of generating a force of up to 0.3 Kg, creating a suction effect. It is an area of air bubbles that, when compressed, facilitate the opening of the lumen for the passage of food towards the pharynx.

Alternatively, a bulb is arranged at the second end of the tube, said bulb being configured to, in an operating condition of the device, be located in the space of Donders and seal said space hermetically or in an airtight manner, to prevent pressure leaks during measurement of the same, making it much more accurate. Moreover, this bulb makes it so that the muscle pressure of the tongue does not modify the measurement of the manometer.

Preferably, the bulb is made from a flexible, soft-touch, unbreakable, and biologically compatible polymeric material.

In another embodiment or indication of use, the device can be used to determine the pressure in the nasopharyngeal space (EPP, EpiPharyngeal Pressure) by measuring the inhalation/exhalation pressure in the nasal cavities of a user's nose, thus becoming a device for determining the nasopharyngeal pressure (EPPI, EpiPharyngeal Pressure Index), wherein the first end of the tube can be connected to the conduit and/or to the pressure gauge, whereas the second end is open to the atmosphere and is intended to be at least partially introduced into a user's nostril where, in an operating condition of this device, the second end is at least partially introduced into one of the nostrils, being coupled in an airtight manner in said nostril, such that the pressure gauge detects the inhalation pressure in the nasopharyngeal space and sends the data on the detection to the processing unit for treatment and analysis.

In an additional embodiment, the foregoing device is fitted to a mask of the type intended to simultaneously cover the user's mouth and nose, such as, for example, FFP2 and FFP3 masks or the like. Even more preferably, the tube can be coupled to the mask by suitable means, for example, passing through the mask through a hole provided therein. This may be useful for measuring breathing during use of these masks which have become popular with the COVID-19 pandemic.

Preferably, a plug is releasably coupled to the second end of the tube, said plug being configured to block in an airtight manner the nasopharyngeal cavity such that, in an operative condition of the device, the plug blocks the nostril so that fluid communication between the pressure gauge and the nostril and/or the nasopharyngeal space is established only through the tube, making the measurement of inhalation pressure more accurate.

The material from which the plug is made must block the nostril without modifying the shape of the tissue inside said nostril so as not to affect the measurement. Therefore, said material should be selected from mouldable, non-porous, soft-touch polymers. According to the foregoing, the connection is completely passive and does not modify the nostril in any way since a soft sponge or similar is used which obstructs without effect on the tissues of the nose.

In this embodiment, the device enables, in addition to measuring and determining pressure in the nasopharyngeal space (EPP) located behind the nasal septum, measuring the pressure/inhalation capacity for each of the nostrils and analysing how the application of a nose decongestant substance affects the ability to inhale/expel air, as well as the effect of a mask covering a user's mouth and nose. In this sense, the device must first be used in one nostril, taking the measurement, and then the device is to be used in the other nostril, taking the measurement. Later, after having applied a dilator, decongestant or some other substance in the nostrils, or having put on the mask, the measurements can be repeated and compared with the initial measurements in order to analyse the effects.

The different embodiments of the described device consist of innovative structural and constituent features heretofore unknown, reasons which, taken together with its usefulness, provide it with sufficient grounds for obtaining the requested exclusivity privilege.

### BRIEF DESCRIPTION OF THE DRAWINGS

To complement the description provided herein, and for the purpose of helping to make the features of the invention more readily understandable, said description is accompanied by a set of drawings constituting an integral part of the same, which by way of illustration and not limitation represents the following:

FIG. 1 shows a general view of the device object of the invention

FIG. 2 shows an exploded view of the components of the device

FIG. 3 shows a detailed view of the components of the electronic card

FIG. 4 shows an alternative embodiment of the device for measuring vacuum pressure in the space of Donders or inhalation pressure in the nasopharyngeal space.

### LIST OF REFERENCES AND FIGURES

1. Intraoral screen
2. Base
3. Conduit
4. Pressure gauge
5. Impermeable membrane
6. Electronic module
7. Wireless connection means
8. Power supply means
9. Data connection port
10. Casing
30. Tube
31. First end of the tube
32. Second end of the tube
33. Bulb
34. Plug

### PREFERRED EMBODIMENT OF THE INVENTION

In the following detailed description of the preferred embodiments, reference is made to the accompanying drawings of this specification and wherein specific preferred embodiments in which the invention may be implemented are shown by way of illustration. These embodiments are described in sufficient detail to enable those skilled in the art to implement the invention, and it is understood that other embodiments may be used and logical structural, mechanical, electrical, and/or chemical changes may be made without departing from the scope of the invention. To avoid details that are not needed by those skilled in the art to implement the detailed description, it should therefore not be taken in a limiting sense.

Specifically, a device is disclosed for determining and modulating the tongue position based on the measurement of vacuum pressure in an oral cavity, which is generated by the tongue movement during the user's swallowing phase, the device comprising:
an intraoral screen 1 intended to, in use or operation, be arranged in the oral cavity, plugging the dental interstices;
a base 2 able to be connected to the intraoral screen 1 through at least one conduit 3, configured to, in a condition of use or operation of the device, establish fluid communication between the inside of the oral cavity and a pressure gauge 4 able to be coupled to the base 2, intended to measure the vacuum pressure produced by swallowing;
and an electronic module 6 able to be coupled to the base 2, said electronic module 6 being in data communication with the pressure gauge 4, and comprising a processing unit configured to obtain data on the tongue position based on the measurement of vacuum pressure in the oral cavity.

In this way, a device capable of determining a user's tongue position is obtained, based on the measurement of the internal pressure of his or her intraoral cavity, thanks to a pressure gauge 4 and the subsequent processing of the data obtained by the same, by means of an electronic module 6.

In a preferred embodiment, the intraoral screen 1 comprises at least one connection end 1A, to which the conduit 3 can be connected, to achieve fluid communication between the inside of the oral cavity and the pressure gauge 4, when the device is in use, measuring the pressure after the user's swallowing process, for example, when swallowing saliva

Preferably, the pressure gauge 4 is a manometer. A person skilled in the art will understand that the term pressure gauge 4 includes all sensitive devices for measuring pressure in the oral cavity and that may be applicable within the context of the invention.

In a preferred embodiment, the intraoral screen 1 will be made of a hydrophobic elastic polymeric material, preferably silicone for intraoral use, so as to achieve adaptability to the morphology of the user's oral cavity and thus seal the oral cavity and prevent air from entering therein when the vacuum has been generated during swallowing.

The preferred position in operation of the intraoral screen 1 will be between the inner portion of the lips and the front portion of the teeth of the user's oral cavity.

Moreover, the electronic module 6 comprises wireless communication means 7, configured to transmit, based on the measurement of the vacuum pressure in the oral cavity, the data on the tongue position towards an external terminal and/or receive information from said external terminal. In such a way that information can be exchanged between the device object of the present invention and said external terminal.

In another preferred embodiment, the device comprises a data connection port 9, able to be coupled to the base 2, which will be able to be connected to the electronic module 6, and wherein the data connection port 9 is configured to transmit data on the tongue position, obtained by measuring the vacuum pressure in the oral cavity, towards the external terminal and/or receive information from said terminal. As a result, information is also exchanged in person, through the direct connection between the data connection port 9 and the external terminal.

As previously mentioned, the electronic module 6 comprises a processing unit configured to obtain data on the tongue position based on the measurement the vacuum pressure in the oral cavity. Such that, given a vacuum pressure established as a reference, for example, 15 m Bar (millibar), the processing unit determines whether the tongue position is correct based on the measurement obtained by the pressure gauge 4 of the vacuum pressure generated in the oral cavity and/or also through the time said negative pressure is maintained.

The processing unit can monitor the position of the tongue, as well as the time that the negative pressure is maintained, through the continuous measurement performed by the pressure gauge 4. For example, if the reference vacuum pressure is 15 m Bar, and the measured pressure is 10 m Bar, this could be insufficient for a proper tongue position, and/or it is also possible that, by having reached the reference pressure, said negative pressure has been sustained for less time than necessary.

According to the last two embodiments, the device comprises both wireless and direct connection communication means for communicating with external devices, successfully exchanging data about the pressure measured in the oral cavity with external terminals, facilitating the processing and treatment of said data.

In a preferred embodiment, the electronic module 6 comprises signalling means configured for emitting a warning signal, wherein the processing unit of the electronic module 6 is configured for, based on the measurement of the negative pressure in the oral cavity, determining an inadequate position of the tongue and transmitting a warning signal through the signalling means.

These signalling means can be in the form of a speaker for sending an acoustic signal, and/or an LED bulb for sending a visual signal, and/or a vibrating mechanism, such as those in smartphones, for sending a vibrating signal, for warning the user of a poor positioning of the tongue inside the oral cavity.

The device comprises power supply means 8, coupleable to the base 2, and configured for supplying power to the electronic module 6 and/or the pressure measurement means 4. Preferably the power supply means 8 will consist of a rechargeable battery detachable from the base 2, such as, for example, a rechargeable battery similar to that of AirPods® by Apple®, or rechargeable batteries of similar devices.

Likewise, in non-illustrated alternative embodiments, the device of the invention comprises additional pressure gauges 4 intended for being coupled to one or more conduits 3 or to other similar conduits (not shown) distributed across the intraoral shield 1. The purpose of arranging these additional pressure gauges 4 is to measure the vacuum pressure in the intraoral compartments, for example, at different points of the intraoral cavity. These additional pressure gauges 4 are coupled to additional conduits 3 suitably distributed across the intraoral shield 1 and are not necessarily arranged at the base 2, although said additional pressure gauges 4 must be in data communication by suitable means with the electronic module 6 for transmitting to the latter the vacuum pressure measurements taken at different points by each of said additional pressure gauges 4.

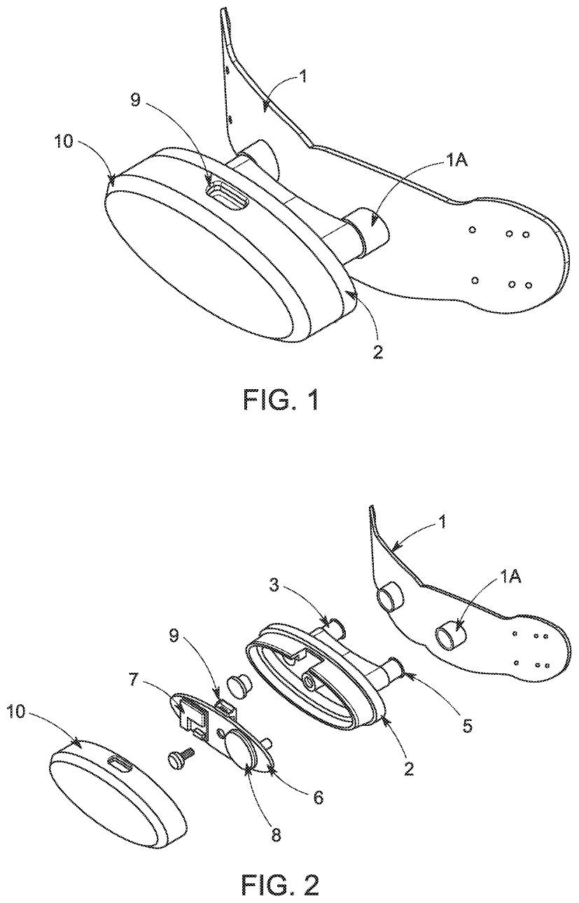

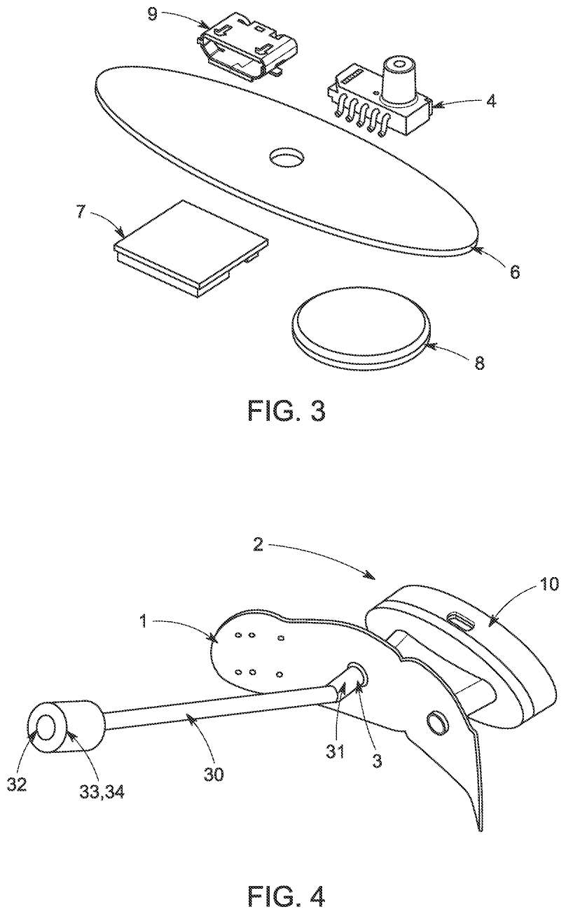

As observed in FIG. 1, in a preferred embodiment, the device comprises a casing 10 coupleable to the base 2 and configured for protecting the electronic module 6 and/or the pressure gauge 4 and/or the data communication port 9 and/or the power supply means 8, wherein the casing 10 comprises at least one opening for accessing the data communication port 9, such that the casing 10 protects the mentioned components from external agents.

Moreover, the device comprises at least one detachable membrane 5, preferably made of an impermeable material, such as GoreTex®, arranged inside the conduit 3 and configured for preventing the passage of fluids through said conduit 3 which may come into contact with the pressure gauge 4 and/or the electronic module 6, causing their deterioration or malfunction.

As observed in FIG. 4, in an alternative embodiment, the device comprises a tube (30) which can be connectable or be connected in a fixed manner at a first end (31) to the conduit (3) and/or to the pressure gauge (4), also comprising a second end (32), which is opposite the first end (31), said second end being open to the atmosphere, such that in an operating condition of the device, the second end (32) of the tube (30) is arranged in a tight manner in the space of Donders between the tongue and the palate of the user, establishing fluid communication between said space of Donders and the pressure gauge (4), such that the pressure gauge (4) detects a pressure which is generally vacuum pressure in said space and sends the data about this detection to the processing unit for treatment.

As seen in FIG. 4, at the second end (32) of the tube (30) there is coupled either in a fixed or in a releasable manner a bulb (33) configured for being located in the space of Donders and sealing said space in a tight manner in an operating condition of the device for preventing pressure leakages that may affect the measurements taken by the device.

The bulb (33) is preferably made from a soft-touch, unbreakable, and biologically compatible flexible polymer material for preventing irritations with parts of the oral cavity with which it comes into contact.

Additionally, by means of the present invention it is possible to take measurement/perform an estimation of the suction pressure in the nasopharyngeal space of the nose of a user.

In this sense, in an operating condition of the device, the second end (32) of the tube (30) is configured for being introduced at least partially into one of the nasal fossae of a user, being arranged in a tight manner in said fossa, such that the pressure gauge (4) detects changes in pressure in the nasopharyngeal space (negative changes during inhalation, positive changes during exhalation) when the air enters and exits through the free nasal fossa, and sends such data about the detection to the processing unit for subsequent treatment.

As seen in FIG. 4, at the second end (32) of the tube (30) there is arranged a plug (34) configured for blocking the nasal fossa into which the second end (32) is introduced such that in the operating condition of the device, the plug (34) blocks the nasal fossa such that fluid communication between the pressure gauge (4) and the free nasal fossa, passing through the nasopharyngeal space, is established only through the tube (30).

It is important to take into account that the bio-functional system also includes the nose, which is characterised by its position between the valves (velopharyngeal sphincter) and the nasal entry.

The nasopharynx is adjacent to the valve; this space is divided by the nasal septum into two channels, nasal cavities, the outer openings of which form the two nasal fossae. From the nasal fossa to the nasopharyngeal space directly adjacent to the valve, there are, therefore, two separate functional spaces. The invention takes advantage of this fact to determine the pressure, and/or alternatively, flow of breathable air.

The left and right halves of the nose can be used separately as a channel to measure pressure in the nasopharyngeal space, which is located next to both halves of the nose, behind the septum.

If the valve is closed on one side and half of the nose behind it is understood to be a pressure conducting channel, a line can be connected in a fluid manner to the pressure gauge (4) at the mid-point of the closed nose. This pressure line measures the pressure existing at the end of the nasal side in the nasopharyngeal space.

Therefore, if the oral cavity is closed by the up-lock condition and the valve of the nasal side connected to the measurement tube (30) is also closed, that is, the second end (32) of the tube (30) is connected in a tight manner in the nasal fossa, breathable air must flow exclusively to the pressure gauge through the tube (30).

A mask (not shown) can be used as part of the device and is configured for simultaneously covering the mouth and nose of the user, the tube (30) being coupleable to the mask by suitable means.

The difference in pressure between the environment and the nasopharyngeal space, that is, the rear end of both nasal sides, will be proportional to the resistance of the side nasal, according to Ohm's law for linear flow. In this sense, it is assumed that a test subject generates a constant air flow for breathing at rest. When breathing with normal intensity and frequency, it requires a greater difference in pressure with a greater flow resistance of the nasal membranes through which the flow arrives.

This can be determined by measuring the amplitude of pressure in the nasopharyngeal space, which must increase with flow resistance of the nose, assuming a constant flow while breathing at rest.

Taking this measurement sequentially on each side of the nose yields result that is useful for diagnostic purposes, being obtained in little time by means of the sequential measurement of the difference in pressure in both nasal fossae, determining pressure in the nasopharyngeal space.

Measurement contour conditions are kept constant by means of the up-lock manoeuvre), such that the secondary air is excluded through the oral cavity towards the nasopharynx. By incorporating the use of the up-lock activator during measurements, pressure in the nasopharynx being caused by a secondary flow through the mouth to the throat is ruled out.

Likewise, the present invention proposes a connectable external terminal in communication with the device of the preceding embodiments, which is configured for receiving data about the position of the tongue obtained by the device, and/or data about the suction pressure in the nasopharyngeal space, and/or determining which of the nasal fossae offers the greatest air suction resistance as described heretofore, wherein the external terminal comprises processing means configured for determining whether or not the position of the tongue is correct based on the obtained data about the position of the tongue, and/or whether the suction pressure in the nasopharyngeal space, and/or the aspiration resistance by each of the nasal fossae, and generating a warning signal through said external terminal for the user of the device of the preceding embodiments.

Having sufficiently described the nature of the present invention, as well as the ways of implementing it, it is not considered necessary to extend its explanation for any person skilled in the state of the art to understand its scope and the advantages which derive from it, specifying that, within its essential nature, it can be carried out in other embodiments that differ in detail from the one provided by way of example, and which are also covered by the requested protection, provided that they do not alter, change or modify its fundamental principle.

1. A device for assessing a correct position of a user's tongue, comprising an intraoral shield (1) intended for being arranged in the oral cavity of the user and configured to block interdental spaces of the teeth in said oral cavity, a conduit (3) connectable at a first end to the intraoral shield (1) and at a second end to a pressure gauge (4), said pressure gauge (4) in turn being couplable to a base (2), wherein the conduit (3) is configured to establish fluid communication between the interior of the oral cavity and the pressure gauge (4) and said pressure gauge (4) is configured to measure the negative pressure generated in the oral cavity due to deglutition, and an electronic module (6) couplable to the base (2), said electronic module (6) being in data communication with the pressure gauge (4) and comprising a processing unit configured to assess the correct position of the tongue by comparing the data of the pressure measured by the pressure gauge (4) and a pre-established pressure; the device characterised in that it comprises a tube (30) connectable at a first end (31) to the conduit (3) and comprising a second end (32), opposite the first end (31), wherein the second end (32) is configured to be arranged in tight manner in the Donders space located between the tongue and the palate of the user, wherein at the second end (32) of the tube (30) is provided a bulb (33) configured to be located in the space of Donders and sealing said space in a tight manner, such that the pressure gauge (4) detects a vacuum pressure in said Donders space and sends the data about this detection to the processing unit for the assessment of the correct position of the tongue.

2. (canceled)

3. The device according to claim 1, wherein the intraoral shield (1) is made from a hydrophobic elastic polymer material.

4. The device according to claim 1, wherein the intraoral shield (1) is configured to be interposed between an inner part of the lips and a frontal part of the teeth in the oral cavity.

5. The device according to claim 1, wherein the electronic module (6) comprises wireless communication means (7) configured for transmitting data about the vacuum pressure in the oral cavity to an external terminal and/or receiving information from said external terminal.

6. The device according to claim 1, comprising a data communication port (9) couplable, to the base (2) and connectable to the electronic module (6), wherein the data communication port (9) is configured for transmitting data about the vacuum pressure in the oral cavity to the external terminal and/or receiving information from said external terminal.

7. The device according to claim 1, wherein the electronic module (6) comprises signalling means configured for emitting a warning signal, wherein the processing unit of the electronic module (6) is configured for, based on the position of the tongue determined from the measurement of the vacuum pressure, determining an inadequate position of the tongue and transmitting a warning signal through the signalling means.

8. The device according to claim 1, comprising power supply means (8) arranged in the base (2), configured for supplying power to the electronic module (6) and/or the pressure gauge (4).

9. The device according to claim 8, wherein the power supply means (8) comprise a rechargeable battery detachable from the base (2).

10. The device according to claim 8, comprising a casing (10) coupleable to the base (2), configured to protect the electronic module (6) and/or the pressure gauge (4) and/or the data communication port (9) and/or the power supply means (8), wherein the casing (10) comprises an opening for accessing the data communication port (9).

11. The device according to claim 1, comprising at least one membrane (5) arranged inside the conduit (3), configured to prevent the passage of fluids through the conduit (3).

12. The device according to claim 11, wherein the membrane (5) is impermeable and interchangeable.

13. The device according to claim 1, comprising one or more pressure gauges (4) coupled to one or more conduits (3).

14. The device according to claim 1, wherein the second end (32) of the tube (30) is further configured to be introduced at least partially into one of the nasal fossae of the nose of a user arranged in a tight manner in the nasal fossae, such that the pressure gauge (4) detects the suction pressure in the nasopharyngeal space and sends data about the detection to the processing unit.

15. The device according to claim 14, comprising a mask configured for simultaneously covering the mouth and the nose of the user, the tube (30) being couplable to the mask.

16. The device according to 14 further comprises a plug (34) arranged at the second end (32) of the tube (30) configured to block the nasal fossa in such a way that the air in said fossa flows through only the tube (30).

17. (canceled)

18. The device according to claim 15 further comprises a plug (34) arranged at the second end (32) of the tube (30) configured to block the nasal fossa in such a way that the air in said fossa flows through only the tube (30).
